(19) United States
(12) Patent Application Publication
(10) Pub. No.: US 2011/0060118 A1
(43) Pub. Date: Mar. 10, 2011

(54) PROCESS FOR THE SYNTHESIS OF FLUORINATED ETHERS OF AROMATIC ACIDS

(75) Inventors: JOACHIM C. RITTER, Wilmington, DE (US); Kenneth Gene Moloy, Hockessin, DE (US); Joel M. Pollino, Elkton, MD (US); Surbhi Mahajan Du, Newark, DE (US)

(73) Assignee: E. I. DU PONT DE NEMOURS AND COMPANY, Wilmington, DE (US)

(21) Appl. No.: 12/874,497

(22) Filed: Sep. 2, 2010

Related U.S. Application Data
(60) Provisional application No. 61/239,106, filed on Sep. 2, 2009.

(51) Int. Cl. C08G 63/66; C07C 51/367

(52) U.S. Cl. 528/361; 562/474

(57) ABSTRACT

Fluorinated ethers of aromatic acids are produced from halogenated aromatic acids in a reaction mixture containing a copper (I) or copper (II) source and a Schiff base ligand that coordinates to copper. The fluorinated ethers of aromatic acids made using the process described herein can be applied to, e.g., fibers, yarns, carpets, garments, films, molded parts, paper and cardboard, stone, and tile to impart soil, water and oil resistance. By incorporating the fluorinated ethers of aromatic acids, or diesters thereof, into polymer backbones, more lasting soil, water and oil resistance, as well as improved flame retardance, can be achieved.

This application claims priority under 35 U.S.C. §119(e) from, and claims the benefit of, U.S. Provisional Application No. 61/239,106, filed Sep. 2, 2009, which is by this reference incorporated in its entirety as a part hereof for all purposes.

### TECHNICAL FIELD

This invention relates to the manufacture of fluorinated ethers of aromatic acids, or hydroxy aromatic acids, which are valuable for a variety of purposes such as use as surfactants, intermediates or as monomers to make polymers.

### BACKGROUND

Fluorinated organic compounds have been used in a wide variety of applications, for example, in surface treatments, as intermediates in the synthesis of, e.g. pharmaceuticals, and as monomers in the synthesis of polymers with highly valued properties. In particular, as compounds or as components of polymers, they are used to impart soil, water and oil resistance, and improved flame retardancy to materials, especially in fiber-related industries. Generally, the fluorinated compounds are applied as a topical treatment, but their effectiveness decreases over time because of material loss resulting from wear and washing.

A need thus remains to provide polymeric materials that have improved, more durable soil and oil resistance.

### SUMMARY

The disclosures herein include new fluorinated ethers of aromatic acids, processes for the preparation of a fluorinated ether of an aromatic acid, processes for the preparation of products into which such a fluorinated ether can be converted, the use of such processes, and the products obtained and obtainable by such processes.

One embodiment of the processes hereof provides a process for preparing a fluorinated ether of an aromatic acid, the ether being represented by the structure of the following Formula I:

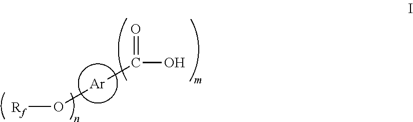

wherein Ar is a C₆˜C₂₀ monocyclic or polycyclic aromatic nucleus, n and m are each independently a nonzero value, n+m is less than or equal to 8, and wherein R_{f} is a fluorinated alkyl, alkaryl, aralkyl or aryl group, optionally containing one or more ether linkages —O—, with the proviso that R_{f} is not attached to the ether oxygen in Formula I via a CF₂ group or a CF₂CH₂CH₂ group, comprising:

(a) contacting a halogenated aromatic acid that is represented by the structure of the following Formula II:

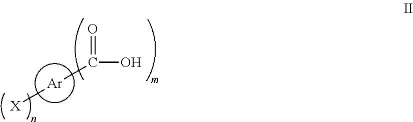

wherein each X is independently Cl, Br, or I, and Ar, n and m are as set forth above, with
(i) a total of from about n+m to about n+m+1 equivalents of the alcoholate R_{f}O⁻M⁺ (wherein M is Na or K) per equivalent of halogenated aromatic acid, in a polar aprotic solvent or in R_{f}OH as a solvent;
(ii) a copper (I) or copper (II) source; and
(iii) a ligand that coordinates to copper, wherein the ligand comprises a Schiff base;
   to form a reaction mixture;

(b) heating the reaction mixture to form the m-basic salt of the product of step (a), as represented by the structure of the following Formula III:

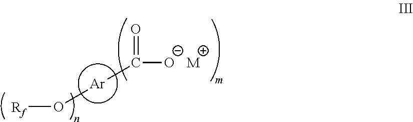

(c) optionally, separating the Formula III m-basic salt from the reaction mixture in which it is formed; and

(d) contacting the Formula III m-basic salt with acid to form therefrom a fluorinated ether of an aromatic acid.

Another embodiment of this invention provides a process for preparing a compound, monomer, oligomer or polymer by preparing a fluorinated ether of an aromatic acid that is described by the structure of Formula I, and then subjecting the ether so produced to a reaction (including a multi-step reaction) to prepare therefrom a compound, monomer, oligomer or polymer.

It has been found that by incorporating fluorinated aromatic diesters into polymer backbones, more lasting soil, water and oil resistance, as well as improved flame retardance, can be achieved.

### DETAILED DESCRIPTION

This disclosure provides a process for preparing a fluorinated ether of an aromatic acid, the ether being represented by the structure of the following Formula I:

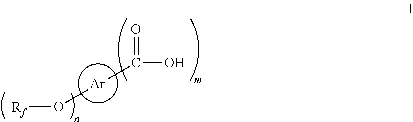

wherein Ar is a C₆˜C₂₀ monocyclic or polycyclic aromatic nucleus, n and m are each independently a nonzero value, n+m is less than or equal to 8, and wherein R_{f} is a fluorinated alkyl, alkaryl, aralkyl or aryl group, optionally containing one or more ether linkages —O—, with the proviso that R_{f} is not attached to the ether oxygen in Formula I via a CF₂ group or a CF₂CH₂CH₂ group, comprising:

(a) contacting a halogenated aromatic acid that is represented by the structure of the following Formula II:

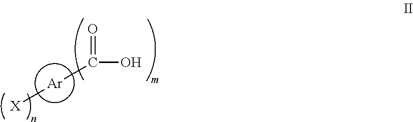

wherein each X is independently Cl, Br or I, and Ar, n and m are as set forth above, with
(i) a total of from about n+m to about n+m+1 equivalents of the alcoholate R_{f}O⁻M⁺ (wherein M is Na or K) per equivalent of halogenated aromatic acid, in a polar aprotic solvent or in R_{f}OH as a solvent;
(ii) a copper (I) or copper (II) source; and
(iii) a ligand that coordinates to copper, wherein the ligand comprises a Schiff base;
   to form a reaction mixture;

(b) heating the reaction mixture to form the m-basic salt of the product of step (a), as is represented by the structure of the following Formula III:

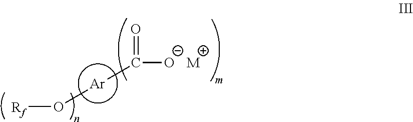

(c) optionally, separating the Formula III m-basic salt from the reaction mixture in which it is formed; and

(d) contacting the Formula III m-basic salt with acid to form therefrom a fluorinated ether of an aromatic acid.

As used herein, the term “alkyl” denotes a univalent group derived from an alkane by removing a hydrogen atom from any carbon atom: —CₓH₂ₓ₊₁ where x≧1.

As used herein, the term “aryl” denotes a univalent group whose free valence is to a carbon atom of an aromatic ring.

As used herein, the term “aralkyl” denotes an alkyl group which bears an aryl group. One such example is the benzyl group, i.e., the radical,

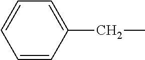

As used herein, the term “alkaryl” denotes an aryl group which bears an alkyl group. Some examples are the 4-methylphenyl radical,

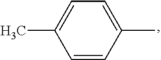

the mesityl group (i.e., 2,4,6-trimethylphenyl) and the 2,6-diisopropylphenyl group (i.e., the (CH₃CHCH₃)₂C₆H₃-radical).

Examples of R_{f} include without limitation:

CF₃(CF₂)ₐ(CH₂)_{b}— wherein a=an integer from 0 to 15 and b=1, 3 or 4;

HCF₂(CF₂)_{c}(CH₂)_{d}— wherein c=an integer from 0 to 15 and d=1, 3, or 4;

CF₃CF₂CF₂OCFHCF₂(OCH₂CH₂)ₑ— and CF₃CF₂CF₂OCF₂CF₂(OCH₂CH₂)ₑ —, wherein e=an integer from 1 to 12;

(CF₃)₂CH—,

(CF₃CF₂CFH)(F)(CF₃)C—,

(CF₃CF₂CFH)(F)(CF₃)CCH₂—,

(CF₃)₂(H)C(CF₃CF₂)(F)C —, and

(CF₃)₂(H)C(CF₃CF₂)(F)CCH₂—; and

pentafluorophenyl.

In Formulae I, II and III, Ar is a C₆˜C₂₀ monocyclic or polycyclic aromatic nucleus; n and m are each independently a nonzero value and n+m is less than or equal to 8; and in Formula II, each X is independently Cl, Br or I.

The radical denoted by

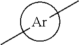

is an n+m valent C₆˜C₂₀ monocyclic or polycyclic aromatic nucleus formed by the removal of n+m hydrogens from different carbon atoms on the aromatic ring, or on the aromatic rings when the structure is polycyclic. The radical “Ar” may be substituted or unsubstituted; when unsubstituted, it contains only carbon and hydrogen.

One example of a suitable Ar group is phenylene, as shown below, wherein n=m=1.

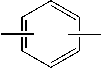

A preferred Ar group is shown below, wherein n=m=2.

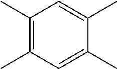

An “m-basic salt”, as the term is used herein, is the salt formed from an acid that contains in each molecule m acid groups having a replaceable hydrogen atom.

Various halogenated aromatic acids, to be used as a starting material in the process of this invention, are commercially available. For example, 2-bromobenzoic acid is available from Aldrich Chemical Company (Milwaukee, Wis.). It can be synthesized, however, by oxidation of bromomethylbenzene as described in Sasson et al, Journal of Organic Chemistry (1986), 51(15), 2880-2883. Other halogenated aromatic acids that can be used include without limitation 2,5-dibromobenzoic acid, 2-bromo-5-nitrobenzoic acid, 2-bromo-5-methylbenzoic acid, 2-chlorobenzoic acid, 2,5-dichlorobenzoic acid, 2-chloro-3,5-dinitrobenzoic acid, 2-chloro-5-methylbenzoic acid, 2-bromo-5-methoxybenzoic acid, 5-bromo-2-chlorobenzoic acid, 2,3-dichlorobenzoic acid, 2-chloro-4-nitrobenzoic acid, 2,5-dichloroterephthalic acid, 2-chloro-5-nitrobenzoic acid, 2,5-dibromoterephthalic acid, and 2,5-dichloroterephthalic acid, all of which are commercially available. Preferably, the halogenated aromatic acid is 2,5-dibromoterephthalic acid or 2,5-dichloroterephthalic acid.

Other halogenated aromatic acids useful as a starting material in the process of this invention include those shown in the left column of the table below, wherein X═Cl, Br or I, and wherein the corresponding ether of an aromatic acid produced therefrom by the process of this invention is shown in the right column:

| (COOH)ₘ—Ar—(X)ₙ | (COOH)ₘ—Ar—(OR_{f})ₙ |
|---|---|
| 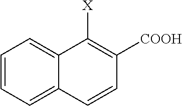 | 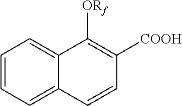 |
| 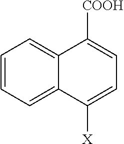 | 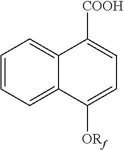 |
| 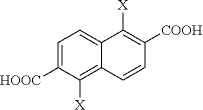 | 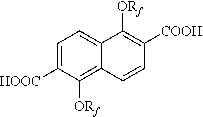 |
| 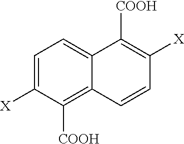 | 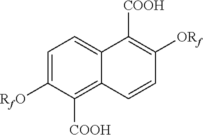 |
| 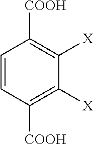 | 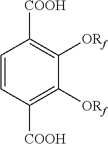 |
| 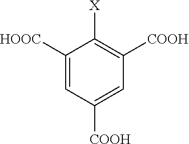 | 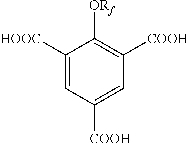 |
| 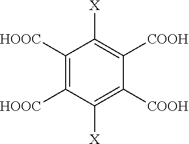 | 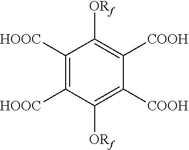 |
| 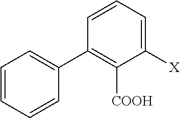 | 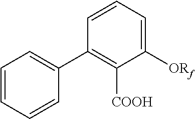 |
| 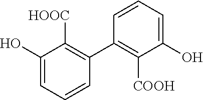 | 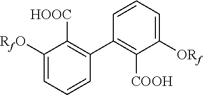 |

In step (a), a halogenated aromatic acid is contacted with the alcoholate R_{f}O⁻M⁺, wherein R_{f} is as defined above and M is Na or K, in a polar aprotic solvent or in R_{f}OH as a solvent; a copper (I) or copper (II) source; and a ligand that coordinates to copper, wherein the ligand comprises a Schiff base.

The alcohol may be R_{f}OH, which is preferred, or it may be an alcohol that is not more acidic than R_{f}OH. Examples of suitable alcohols include without limitation methanol, ethanol, i-propanol, i-butanol, and phenol, with the proviso that the alcohol is not more acidic than R_{f}OH.

The solvent may also be a polar protic or polar aprotic solvent or a mixture of protic or polar aprotic solvent. A polar solvent, as used herein, is a solvent whose constituent molecules exhibit a nonzero dipole moment. A polar protic solvent, as used herein, is a polar solvent whose constituent molecules contain an O—H or N—H bond. A polar aprotic solvent, as used herein, is a polar solvent whose constituent molecules do not contain an O—H or N—H bond. Non-limiting examples of polar solvents other than an alcohol suitable for use herein include tetrahydrofuran, N-methylpyrrolidone, dimethylformamide, and dimethylacetamide.

In step (a), a halogenated aromatic acid is preferably contacted with a total of from about n+m to n+m+1 equivalents of the alcoholate RO⁻M⁺ per equivalent of halogenated aromatic acid. Between m and m+1 equivalents is used for forming the m-basic salt and between n and n+1 equivalents is used for the displacement reaction. It is preferred that the total amount of alcoholate not exceed m+n+1. It is also preferred that the total amount of alcoholate not be less than m+n in order to avoid reduction reactions. One “equivalent” as used in this context is the number of moles of alcoholate RO⁻M⁺ that will react with one mole of hydrogen ions; for an acid, one equivalent is the number of moles of acid that will supply one mole of hydrogen ions.

As mentioned above, in step (a), the halogenated aromatic acid is also contacted with a copper (I) or (II) source in the presence of a Schiff base ligand that coordinates to copper. The copper source and the ligand may be added sequentially to the reaction mixture, or may be combined separately (for example, in a solution of water or acetonitrile) and added together.

The copper source is a Cu(I) salt, a Cu(II) salt, or mixtures thereof. Examples include without limitation CuCl, CuBr, CuI, Cu₂SO₄, CuNO₃, CuCl₂, CuBr₂, CuI₂, CuSO₄, and Cu(NO₃)₂. The selection of the copper source may be made in relation to the identity of the halogenated aromatic acid used. For example, if the starting halogenated aromatic acid is a bromobenzoic acid, CuCl, CuBr, CuI, Cu₂SO₄, CuNO₃, CuCl₂, CuBr₂, CuI₂, CuSO₄, and Cu(NO₃)₂ will be included among the useful choices. If the starting halogenated aromatic acid is a chlorobenzoic acid, CuBr, CuI, CuBr₂ and CuI₂ will be included among the useful choices. Optionally, prior to step (a), a measured amount (˜0.25 mol of O₂/mol of CuI) may be added to dissolve CuI in the diamine/alcohol solution. CuBr and CuBr₂ are in general preferred choices for most systems. The amount of copper used is typically about 0.1 to about 5 mol % based on moles of halogenated aromatic acid.

The ligand comprises a Schiff base. The term “Schiff base” as used herein denotes a functional group or type of chemical compound containing a carbon-nitrogen double bond with the nitrogen atom connected to an aryl group or an alkyl group but not to hydrogen, such as shown by the structure of Formula IV. It is typically the condensation product of a primary amine and a ketone or aldehyde, produced by a reaction scheme such as the following:

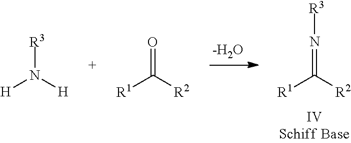

wherein R¹, R² and R³ are each independently selected from substituted and unsubstituted C₁-C₁₆ n-alkyl, iso-alkyl and tertiary alkyl groups; and substituted and unsubstituted C₆-C₃₀ aryl and heteroaryl groups.

In one embodiment, a Schiff base suitable for use herein as the ligand includes a diimine such as described generally by Formula V

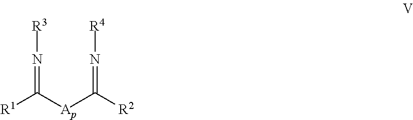

wherein A is selected from the group consisting of

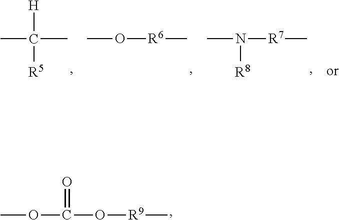

R¹, R², R³ and R⁴ are each independently selected from substituted and unsubstituted C₁-C₁₆ n-alkyl, iso-alkyl and tertiary alkyl groups; and substituted and unsubstituted C₆-C₃₀ aryl and heteroaryl groups;

R⁵ is selected from H, substituted and unsubstituted C₁-C₁₆ n-alkyl, iso-alkyl and tertiary alkyl groups; and substituted and unsubstituted C₆-C₃₀ aryl and heteroaryl groups; and halogen;

R⁶, R⁷, R⁸ and R⁹ are each independently selected from H or a substituted or unsubstituted C₁-C₁₆ n-alkyl, iso-alkyl or tertiary alkyl group; and p=0 or 1.

The term “unsubstituted”, as used with reference to an alkyl or aryl group in a Schiff base as described above, means that the alkyl or aryl group contains no atoms other than carbon and hydrogen. In a substituted alkyl or aryl group, however, one or more O or S atoms may optionally be substituted for any one or more of the in-chain or in-ring carbon atoms, provided that the resulting structure contains no —O—O— or —S—S— moieties, and provided that no carbon atom is bonded to more than one heteroatom.

In another embodiment, a suitable diimine for use herein as the ligand includes N,N′-dimesityl-2,3-diiminobutane (such as described generally by Formula VI)

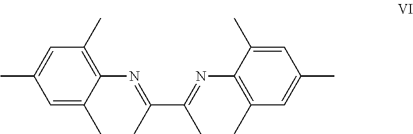

In this instance, p=0, R¹═R²mesityl, and R³ and R⁴ are taken together to form the CH₃—C—C—CH₃ moiety bonded to the two nitrogen atoms.

In a further embodiment, a diimine suitable for use herein as the ligand includes N,N′-di(trifluoromethylbenzene)-2,3-diiminoethane (such as described generally by Formula VII)

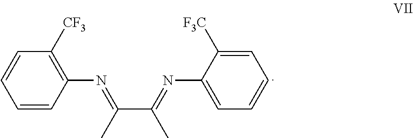

In this instance, p=0, R¹═R²=(trifluoromethyl)benzyl, and R³ and R⁴ are taken together to form the CH₃—C—C—CH₃ moiety bonded to the two nitrogen atoms.

A ligand suitable for use herein may be selected as any one or more or all of the members of the whole population of ligands described by name or structure above.

Various copper sources and ligands suitable for use herein may be made by processes known in the art, or are available commercially from suppliers such as Alfa Aesar (Ward Hill, Mass.), City Chemical (West Haven, Conn.), Fisher Scientific (Fairlawn, N.J), Sigma-Aldrich (St. Louis, Mo.) or Stanford Materials (Aliso Viejo, Calif.).

In various embodiments, the ligand may be provided in an amount of about 1 to about 8, preferably about 1 to about 2, molar equivalents of ligand per mole of copper. In those and other embodiments, the ratio of molar equivalents of ligand to molar equivalents of halogenated aromatic acid may be less than or equal to about 0.1. As used herein, the term “molar equivalent” indicates the number of moles of ligand that will interact with one mole of copper.

In step (b), the reaction mixture is heated to form the m-basic salt as represented by the structure of the following Formula III:

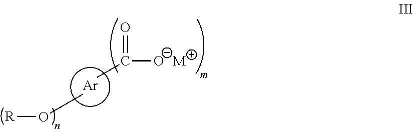

The reaction temperature for steps (a) and (b) is preferably between about 40 and about 120° C., more preferably between about 50 and about 90° C. Typically, the time required for step (a) is from about 0.1 to about 1 hour. The time required for step (b) is typically from about 1 to about 100 hours. Optimal times and temperatures may vary depending on the specific materials. Oxygen may be desirably excluded during the reaction. The solution is typically allowed to cool before optional step (c) and before the acidification in step (d) is carried out.

The m-basic salt of the ether of the aromatic acid is then contacted in step (d) with acid to convert it to the hydroxy aromatic acid product. Any acid of sufficient strength to protonate the m-basic salt is suitable. Examples include without limitation hydrochloric acid, sulfuric acid and phosphoric acid.

In one embodiment, the copper (I) or copper (II) source is selected from the group consisting of CuBr, CuBr₂ and mixtures thereof; the ligand is selected from the group consisting of N,N′-dimesityl-2,3-diiminobutane and N,N′-di(trifluoromethylbenzene)-2,3-diiminoethane; and the copper (I) or copper (II) source is combined with two molar equivalents of the ligand.

The fluorinated ethers of aromatic acids made using the process described herein can be fabricated as fibers, yarns, carpets, garments, films, molded parts, paper and cardboard, stone, and tile to impart soil, water and oil resistance. By incorporating the fluorinated ethers of aromatic acids, or diesters thereof, into polymer backbones, more lasting soil, water and oil resistance, as well as improved flame retardance, can be achieved.

The process described above also allows for effective and efficient synthesis of products made from the resulting fluorinated ethers of aromatic acids such as a compound, a monomer, or an oligomer or polymer thereof. These produced materials may have one or more of ester functionality, ether functionality, amide functionality, imide functionality, imidazole functionality, thiazole functionality, oxazole functionality, carbonate functionality, acrylate functionality, epoxide functionality, urethane functionality, acetal functionality, or anhydride functionality.

A Formula I compound may, as desired, be isolated and recovered as described above. It may also be subjected with or without recovery from the reaction mixture to further steps to convert it to another product such as another compound (e.g. a monomer), or an oligomer or a polymer. Another embodiment of a process hereof thus provides a process for converting a Formula I compound, through one or more reactions, into another compound, or into an oligomer or a polymer. A Formula I compound may be made by a process such as described above, and then may be subjected, for example, to a polymerization reaction to prepare an oligomer or polymer therefrom, such as those having ester functionality or amide functionality, or a pyridobisimidazole-2,6-diyl(2,5-dihydroxy-p-phenylene)polymer.

The compounds of Formula I made by the process disclosed herein, or their diesters, in particular dimethyl esters, can be used in condensation polymerizations to produce fluorinated condensation polymers, e.g., including without limitation polyesters, polyamides, polyimides, and polybenzimidazoles. Representative reactions involving a material of this invention, or a derivative of such material, such as a diester, include, for example, making a polyester from one or more compounds of Formula I and either diethylene glycol or triethylene glycol in the presence of 0.1% of Zn₃(BO₃)₂ in 1-methylnaphthalene under nitrogen, according to the method taught in U.S. Pat. No. 3,047,536 (which is incorporated in its entirety as a part hereof for all purposes). Similarly, a fluorinated ether of aromatic acid is suitable for copolymerization with a dibasic acid and a glycol to prepare a heat-stabilized fluorinated polyester according to the method taught in U.S. Pat. No. 3,227,680 (which is incorporated in its entirety as a part hereof for all purposes), wherein representative conditions involve forming a prepolymer in the presence of titanium tetraisopropoxide in butanol at 200˜250° C., followed by solid-phase polymerization at 280° C. at a pressure of 0.08 mm Hg.

Other diols useful to make from a polyester from a Formula I compound are those that are derived from a fermentation process, and another embodiment of this invention thus involves a process for making from a Formula I compound an oligomer or polymer that further includes a step of providing a diol to such a process from a fermentation process.

A Formula I compound may be converted into a polyamide oligomer or polymer by reaction with a diamine in a process in which, for example, the polymerization takes place in solution in an organic compound that is liquid under the conditions of the reaction, is a solvent for both the Formula I compound and the diamine, and has a swelling or partial salvation action on the polymeric product. The reaction may be effected at moderate temperatures, e.g. under 100° C., and is preferably effected in the presence of an acid acceptor that is also soluble in the chosen solvent. Suitable solvents include methyl ethyl ketone, acetonitrile, N,N-dimethylacetamide dimethyl formamide containing 5% lithium chloride, and N-methyl pyrrolidone containing a quaternary ammonium chloride such as methyl tri-n-butyl ammonium chloride or methyl-tri-n-propyl ammonium chloride. Combination of the reactant components causes generation of considerable heat and the agitation, also, results in generation of heat energy. For that reason, the solvent system and other materials are cooled at all times during the process when cooling is necessary to maintain the desired temperature. Processes similar to the foregoing are described in U.S. Pat. No. 3,554,966; U.S. Pat. No. 4,737,571; and CA 2,355,316.

A Formula I compound may also be converted into a polyamide oligomer or polymer by reaction with a diamine in a process in which, for example, a solution of the diamine in a solvent may be contacted in the presence of an acid acceptor with a solution of the Formula I compound in a second solvent that is immiscible with the first to effect polymerization at the interface of the two phases. The diamine may, for example, be dissolved or dispersed in a water containing base with the base being used in sufficient quantities to neutralize the acid generated during polymerization. Sodium hydroxide may be used as the acid acceptor. Preferred solvents for the diacid(halide) are tetrachloroethylene, methylenechloride, naphtha and chloroform. The solvent for the Formula I compound should be a relative non-solvent for the amide reaction product, and be relatively immiscible in the amine solvent. A preferred threshold of immiscibility is as follows: an organic solvent should be soluble in the amine solvent not more than between 0.01 weight percent and 1.0 weight percent. The diamine, base and water are added together and vigorously stirred. High shearing action of the stirrer is important. The solution of acid chloride is added to the aqueous slurry. Contacting is generally carried out at from 0° C. to 60° C., for example, for from about 1 second to 10 minutes, and preferably from 5 seconds to 5 minutes at room temperature. Polymerization occurs rapidly. Processes similar to the foregoing are described in U.S. Pat. No. 3,554,966 and U.S. Pat. No. 5,693,227.

A fluorinated ether of aromatic acid can also be polymerized with the trihydrochloride-monohydrate of tetraaminopyridine in a condensation polymerization in strong polyphosphoric acid under slow heating above 100° C. up to about 180° C. under reduced pressure, followed by precipitation in water, as disclosed in U.S. Pat. No. 5,674,969 (which is incorporated in its entirety as a part hereof for all purposes); or by mixing the monomers at a temperature from about 50° C. to about 110° C., and then 145° C. to form an oligomer, and then reacting the oligomer at a temperature of about 160° C. to about 250° C. as disclosed in U.S. Provisional Application No. 60/665,737, filed Mar. 28, 2005 (which is incorporated in its entirety as a part hereof for all purposes), published as WO 2006/104974. The polymer that may be so produced may be a pyridobisimidazole-2,6-diyl(2,5-dialkoxy-p-phenylene)polymer or a pyridobisimidazole-2,6-diyl(2,5-diareneoxy-p-phenylene)polymer such as a poly(1,4-(2,5-diareneoxy)phenylene-2,6-pyrido[2,3-d: 5,6-d′]bisimidazole)polymer. The pyridobisimidazole portion thereof may, however, be replaced by any one or more of a benzobisimidazole, benzobisthiazole, benzobisoxazole, pyridobisthiazole and a pyridobisoxazole; and the 2,5-dialkoxy-p-phenylene portion thereof may be replaced by an alkyl or aryl ether of one or more of isophthalic acid, terephthalic acid, 2,5-pyridine dicarboxylic acid, 2,6-naphthalene dicarboxylic acid, 4,4′-diphenyl dicarboxylic acid, 2,6-quinoline dicarboxylic acid, and 2,6-bis(4-carboxyphenyl)pyridobisimidazole, wherein such a fluorinated ether is produced according to the methods disclosed herein.

The polymer prepared in such manner may, for example, contain one or more of the following units:

pyridobisimidazole-2,6-diyl(2,5-dialkoxy-p-phenylene) and/or pyridobisimidazole-2,6-diyl(2,5-diphenoxy-p-phenylene) units;

units selected from the group consisting of pyridobisimidazole-2,6-diyl(2,5-dimethoxy-p-phenylene), pyridobisimidazole-2,6-diyl(2,5-diethoxy-p-phenylene), pyridobisimidazole-2,6-diyl(2,5-dipropoxy-p-phenylene), pyridobisimidazole-2,6-diyl(2,5-dibutoxy-p-phenylene) and pyridobisimidazole-2,6-diyl(2,5-diphenoxy-p-phenylene);

pyridobisthiazole-2,6-diyl(2,5-dialkoxy-p-phenylene) and/or pyridobisthiazole-2,6-diyl(2,5-diphenoxy-p-phenylene) units;

units selected from the group consisting of pyridobisthiazole-2,6-diyl(2,5-dimethoxy-p-phenylene), pyridobisthiazole-2,6-diyl(2,5-diethoxy-p-phenylene), pyridobisthiazole-2,6-diyl(2,5-dipropoxy-p-phenylene), pyridobisthiazole-2,6-diyl(2,5-dibutoxy-p-phenylene) and pyridobisthiazole-2,6-diyl(2,5-diphenoxy-p-phenylene);

pyridobisoxazole-2,6-diyl(2,5-dialkoxy-p-phenylene) and/or pyridobisoxazole-2,6-diyl(2,5-diphenoxy-p-phenylene) units;

units selected from the group consisting of pyridobisoxazole-2,6-diyl(2,5-dimethoxy-p-phenylene), pyridobisoxazole-2,6-diyl(2,5-diethoxy-p-phenylene), pyridobisoxazole-2,6-diyl(2,5-dipropoxy-p-phenylene), pyridobisoxazole-2,6-diyl(2,5-dibutoxy-p-phenylene) and pyridobisoxazole-2,6-diyl(2,5-diphenoxy-p-phenylene); benzobisimidazole-2,6-diyl(2,5-dialkoxy-p-phenylene) and/or benzobisimidazole-2,6-diyl(2,5-diphenoxy-p-phenylene) units;

units selected from the group consisting of benzobisimidazole-2,6-diyl(2,5-dimethoxy-p-phenylene), benzobisimidazole-2,6-diyl(2,5-diethoxy-p-phenylene), benzobisimidazole-2,6-diyl(2,5-dipropoxy-p-phenylene), benzobisimidazole-2,6-diyl(2,5-dibutoxy-p-phenylene) and benzobisimidazole-2,6-diyl(2,5-diphenoxy-p-phenylene);

benzobisthiazole-2,6-diyl(2,5-dialkoxy-p-phenylene) and/or benzobisthiazole-2,6-diyl(2,5-diphenoxy-p-phenylene) units;

units selected from the group consisting of benzobisthiazole-2,6-diyl(2,5-dimethoxy-p-phenylene), benzobisthiazole-2,6-diyl(2,5-diethoxy-p-phenylene), benzobisthiazole-2,6-diyl(2,5-dipropoxy-p-phenylene), benzobisthiazole-2,6-diyl(2,5-dibutoxy-p-phenylene) and benzobisthiazole-2,6-diyl(2,5-diphenoxy-p-phenylene);

benzobisoxazole-2,6-diyl(2,5-dialkoxy-p-phenylene) and/or benzobisoxazole-2,6-diyl(2,5-diphenoxy-p-phenylene) units; and/or

units selected from the group consisting of benzobisoxazole-2,6-diyl(2,5-dimethoxy-p-phenylene), benzobisoxazole-2,6-diyl(2,5-diethoxy-p-phenylene), benzobisoxazole-2,6-diyl(2,5-dipropoxy-p-phenylene), benzobisoxazole-2,6-diyl(2,5-dibutoxy-p-phenylene) and benzobisoxazole-2,6-diyl(2,5-diphenoxy-p-phenylene).

### Examples

The advantageous attributes and effects of the processes hereof may be seen in laboratory examples, as described below. The embodiments of these processes on which the example is based are representative only, and the selection of those embodiments to illustrate the invention does not indicate that conditions, arrangements, approaches, steps, techniques, configurations or reactants not described in the example are not suitable for practicing these processes, or that subject matter not described in the example is excluded from the scope of the appended claims and equivalents thereof.

The meaning of abbreviations is as follows “mL” means milliliter(s), “g” means gram(s), “mmol” means millimole(s), “N” means normal, and “THF” means tetrahydrofuran.

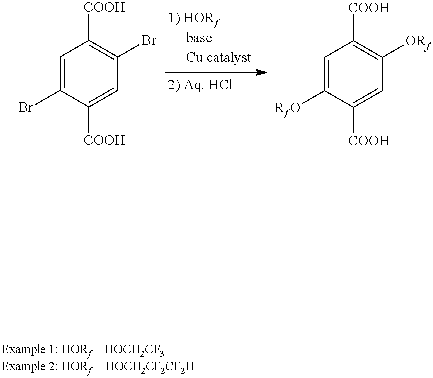

### Example 1

### Preparation of 2,5-bis(2,2,2-trifluoroethoxy)terephthalic acid

To a solution of 8 mL 2,2,2-trifluoroethanol (CF₃CH₂OH) in 15 mL of THF is carefully added 0.19 g (7.9 mmol) of sodium hydride. When gas evolution is complete, 0.488 g (1.5 mmol) of 2,5-dibromoterephthalic acid was added to the solution, followed by addition of a solution of CuBr₂ (0.092 mmol) and N,N′-dimesityl-2,3-diiminobutane

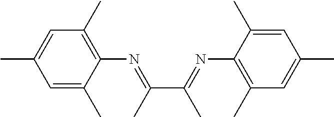

(0.19 mmol) in 1.5 mL of CF₃CH₂OH. The resulting pale blue slurry is heated at 60° C. for four days. Aqueous HCl (1 N) is added to precipitate the product. The product is washed with water, then dissolved in methanol, and the resulting solution is filtered. The methanol is removed under vacuum to give the product 2,5-bis(2,2,2-trifluoroethoxy)terephthalic acid as colorless microcrystals.

### Example 2

### Preparation of 2,5-bis(2,2,3,3-tetrafluoropropoxy)terephthalic acid

A flask is charged with 5 mL of anhydrous THF and 8.1 mmol of sodium hydride. A solution of 1.5 g (11.4 mmol) of 2,2,3,3-tetrafluoropropanol (HCF₂CF₂CH₂OH) in 5 mL of THF is added dropwise. When gas evolution is complete, 2,5-dibromoterephthalic acid (1.51 mmol) is added to the colorless solution. Next, a mixture of CuBr₂ (0.13 mmol) and N,N′-di(trifluoromethylbenzene)-2,3-diiminoethane

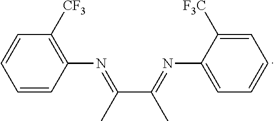

(0.22 mmol) in 0.5 g of HCF₂CF₂CH₂OH is added to the solution. The resulting pale blue slurry is heated at 60° C. for two days. The product 2,5-bis(2,2,3,3-tetrafluoropropoxy)terephthalic acid is isolated by treating the cooled reaction product with 0.5 N HCl, then with water, and washing the precipitate with water.

Each of the formulae shown herein describes each and all of the separate, individual compounds that can be formed in that formula by (1) selection from within the prescribed range for one of the variable radicals, substituents or numerical coefficients while all of the other variable radicals, substituents or numerical coefficients are held constant, and (2) performing in turn the same selection from within the prescribed range for each of the other variable radicals, substituents or numerical coefficients with the others being held constant. In addition to a selection made within the prescribed range for any of the variable radicals, substituents or numerical coefficients of only one of the members of the group described by the range, a plurality of compounds may be described by selecting more than one but less than all of the members of the whole group of radicals, substituents or numerical coefficients. When the selection made within the prescribed range for any of the variable radicals, substituents or numerical coefficients is a subgroup containing (i) only one of the members of the whole group described by the range, or (ii) more than one but less than all of the members of the whole group, the selected member(s) are selected by omitting those member(s) of the whole group that are not selected to form the subgroup. The compound, or plurality of compounds, may in such event be characterized by a definition of one or more of the variable radicals, substituents or numerical coefficients that refers to the whole group of the prescribed range for that variable but where the member(s) omitted to form the subgroup are absent from the whole group.

Where a range of numerical values is recited herein, the range includes the endpoints thereof and all the individual integers and fractions within the range, and also includes each of the narrower ranges therein formed by all the various possible combinations of those endpoints and internal integers and fractions to form subgroups of the larger group of values within the stated range to the same extent as if each of those narrower ranges was explicitly recited. Where a range of numerical values is stated herein as being greater than a stated value, the range is nevertheless finite and is bounded on its upper end by a value that is operable within the context of the invention as described herein. Where a range of numerical values is stated herein as being less than a stated value, the range is nevertheless bounded on its lower end by a non-zero value.

In this specification, unless explicitly stated otherwise or indicated to the contrary by the context of usage, amounts, sizes, ranges and other quantities and characteristics recited herein, particularly when modified by the term “about”, may but need not be exact, and may also be approximate and/or larger or smaller (as desired) than stated, reflecting tolerances, conversion factors, rounding off, measurement error and the like, as well as the inclusion within a stated value of those values outside it that have, within the context of this invention, functional and/or operable equivalence to the stated value.

Where an embodiment of this invention is stated or described as comprising, including, containing, having, being composed of or being constituted by certain features, it is to be understood, unless the statement or description explicitly provides to the contrary, that one or more features in addition to those explicitly stated or described may be present in the embodiment. An alternative embodiment of this invention, however, may be stated or described as consisting essentially of certain features, in which embodiment features that would materially alter the principle of operation or the distinguishing characteristics of the embodiment are not present therein. A further alternative embodiment of this invention may be stated or described as consisting of certain features, in which embodiment, or in insubstantial variations thereof, only the features specifically stated or described are present.

What is claimed is:

1. A process for preparing a fluorinated ether of an aromatic acid, the ether being represented by the structure of the following Formula I: 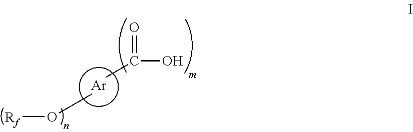
wherein Ar is a C₆˜C₂₀ monocyclic or polycyclic aromatic nucleus, n and m are each independently a nonzero value, n+m is less than or equal to 8, and wherein R_{f} is a fluorinated alkyl, alkaryl, aralkyl or aryl group, optionally containing one or more ether linkages —O—, with the proviso that R_{f} is not attached to the ether oxygen in Formula I via a CF₂ group or a CF₂CH₂CH₂ group, comprising:
(a) contacting a halogenated aromatic acid that is represented by the structure of the following Formula II: 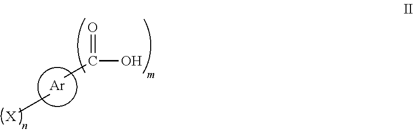
wherein each X is independently Cl, Br or I, and Ar, n and m are as set forth above, with
(i) a total of from about n+m to about n+m+1 equivalents of the alcoholate R_{f}O⁻M⁺ (wherein M is Na or K) per equivalent of halogenated aromatic acid, in a polar aprotic solvent or in R_{f}OH as a solvent;
(ii) a copper (I) or copper (II) source; and
(iii) a ligand that coordinates to copper, wherein the ligand comprises a Schiff base;
to form a reaction mixture;
(b) heating the reaction mixture to form the m-basic salt of the product of step (a), as represented by the structure of the following Formula III: 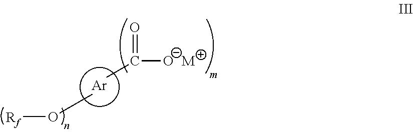
(c) optionally, separating the Formula III m-basic salt from the reaction mixture in which it is formed; and
(d) contacting the Formula III m-basic salt with acid to form therefrom a fluorinated ether of an aromatic acid.

2. A process according to claim 1 wherein R_{f} is selected from the group consisting of:
CF₃(CF₂)ₐ(CH₂)_{b}— wherein a=an integer from 0 to 15 and b 32 1, 3 or 4;
HCF₂(CF₂)_{c}(CH₂)_{d}— wherein c=an integer from 0 to 15 and d=1, 3, or 4;
CF₃CF₂CF₂OCFHCF₂(OCH₂CH₂)ₑ— and
CF₃CF₂CF₂OCF₂CF₂(OCH₂CH₂)ₑ—, wherein e=an integer from 1 to 12;
(CF₃)₂CH—,
(CF₃CF₂CFH)(F)(CF₃)C—,
(CF₃CF₂CFH)(F)(CF₃)CCH₂—,
(CF₃)₂(H)C(CF₃CF₂)(F)C—, and
(CF₃)₂(H)C(CF₃CF₂)(F)CCH₂—; and
pentafluorophenyl.

3. A process according to claim 1 wherein the halogenated aromatic acid is selected from the group consisting of 2-bromobenzoic acid, 2,5-dibromobenzoic acid, 2-bromo-5-nitrobenzoic acid, 2-bromo-5-methylbenzoic acid, 2-chlorobenzoic acid, 2,5-dichlorobenzoic acid, 2-chloro-3,5-dinitrobenzoic acid, 2-chloro-5-methylbenzoic acid, 2-bromo-5-methoxybenzoic acid, 5-bromo-2-chlorobenzoic acid, 2,3-dichlorobenzoic acid, 2-chloro-4-nitrobenzoic acid, 2,5-dichloroterephthalic acid, 2-chloro-5-nitrobenzoic acid, 2,5-dibromoterephthalic acid, and 2,5-dichloroterephthalic acid.

4. A process according to claim 1 wherein, in step (a), a total of about n+m to n+m+1 normal equivalents of R_{f}O⁻M⁻ are added to the reaction mixture per equivalent of the halogenated aromatic acid.

5. A process according to claim 1 wherein the copper source comprises a Cu(I) salt, a Cu(II) salt, or a mixture thereof.

6. A process according to claim 5 wherein the copper source is selected from the group consisting of CuCl, CuBr, CuI, Cu₂SO₄, CuNO₃, CuCl₂, CuBr₂, CuI₂, CuSO₄, Cu(NO₃)₂, and mixtures thereof.

7. A process according to claim 1 wherein the ligand is described by the structure of Formula IV. 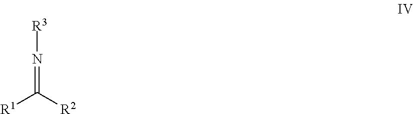
wherein R¹, R² and R³ are each independently selected from substituted and unsubstituted C₁-C₁₆ n-alkyl, iso-alkyl and tertiary alkyl groups; and substituted and unsubstituted C₆-C₃₀ aryl and heteroaryl groups.

8. A process according to claim 1 wherein the ligand is described by the structure of Formula V 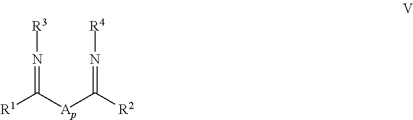
wherein A is selected from the group consisting of 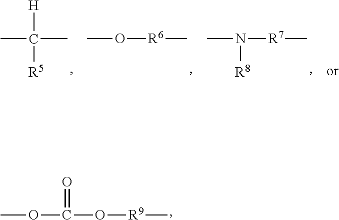
R¹, R², R³ and R⁴ are each independently selected from substituted and unsubstituted C₁-C₁₆ n-alkyl, iso-alkyl and tertiary alkyl groups; and substituted and unsubstituted C₆-C₃₀ aryl and heteroaryl groups;
R⁵ is selected from H, substituted and unsubstituted C₁-C₁₆ n-alkyl, iso-alkyl and tertiary alkyl groups; and substituted and unsubstituted C₆-C₃₀ aryl and heteroaryl groups; and halogen;
R⁶, R⁷, R⁸ and R⁹ are each independently selected from H or a substituted or unsubstituted C₁-C₁₆ n-alkyl, iso-alkyl or tertiary alkyl group; and
p=0 or 1.

9. A process according to claim 8 wherein p=0 and R³ and R⁴ are taken together to form the CH₃—C—C—CH₃ moiety bonded to the two nitrogen atoms.

10. A process according to claim 9 wherein the ligand is N,N′-dimesityl-2,3-diiminobutane or N,N′-di(trifluoromethylbenzene)-2,3-diiminoethane.

11. A process according to claim 1 further comprising a step of combining the copper source with the ligand before adding them to the reaction mixture.

12. A process according to claim 6 wherein the copper source comprises CuBr or CuBr₂.

13. A process according to claim 1 wherein copper is provided in an amount of between about 0.1 and about 5 mol % based on moles of halogenated aromatic acid.

14. A process according to claim 1 wherein the ligand is provided in an amount of between about one and about two molar equivalents per mole of copper.

15. A process according to claim 1 wherein the halogenated aromatic hydroxy acid comprises 2,5-dibromoterephthalic acid or 2,5-dichloroterephthalic acid; the copper source comprises CuBr, CuBr₂ or a mixture of CuBr and CuBr₂; the copper source is provided in an amount of between about 0.1 and about 5 mol % based on moles of halogenated aromatic acid; the ligand is N,N′-dimesityl-2,3-diiminobutane or N,N′-di(trifluoromethylbenzene)-2,3-diiminoethane; and the ligand is provided in an amount of between about one and about two molar equivalents per mole of copper.

16. A process according to claim 1 further comprising a step of subjecting the ether of the aromatic acid to a reaction to prepare therefrom a compound, monomer, oligomer or polymer.

17. A process according to claim 16 wherein a polymer prepared comprises at least one member of the group consisting of pyridobisimidazole, pyridobisthiazole, pyridobisoxazole, benzobisimidazole, benzobisthiazole, and benzobisoxazole moieties.

18. A process according to claim 17 wherein a polymer prepared comprises a fluorinated pyridobisimidazole-2,6-diyl(2,5-dialkoxy-p-phenylene)polymer or a fluorinated pyridobisimidazole-2,6-diyl(2,5-diareneoxy-p-phenylene)polymer.
